(19) United States
(12) Patent Application Publication
(10) Pub. No.: US 2003/0229063 A1
(43) Pub. Date: Dec. 11, 2003

(54) Use of 4-hydroxytestosterone to lower estrogen levels in humans

(76) Inventor: William Charles Llewellyn, Sound Beach, NY (US)

Correspondence Address: William Llewellyn, PO Box 1162, Sound Beach, NY, 11789, US

(21) Appl. No.: 10/163,107

(22) Filed: Jun. 5, 2002

(51) Int. Cl. A61K 31/57

(52) U.S. Cl. 514/178

(57) ABSTRACT

This invention discloses methods of administering 4-hydroxytestosterone in order to lower estrogen levels in humans. As men age, a decline in androgenic hormone levels is typically noted, possibly resulting in muscle mass, bone density and energy loss. This is often accompanied by elevated estrogen to androgen ratio. Various methods have therefore been developed to supplement androgens for men with declining levels and/or correct this ratio. Some such have focused on the use of direct aromatse inhibitors, as a means of lowering levels of estrogen in humans. This invention is an improvement over the use of the aromatase inhibitor 4-hydroxyandrostenedione, in that the subject of this invention is an aromatase inhibitor developed by modifying an active androgen instead of an inactive metabolite. This may be a very advantageous trait for aging men who require a safe and effective way to treat estrogen/androgen imbalance.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

There are numerous endocrine disorders in men characterized by a high ratio of estrogens to androgens. Androgens such as testosterone are responsible for the development and maintenance of male sexual characteristics, including external virilization, sexual maturity at puberty, spermatogenesis, sexual behavior/libido and erectile functioning. They also support bone and muscle tissue growth, and remain vital to ones health and well being throughout life. After physical maturity, men often notice a slow decline in the level of testosterone produced by the body. Dubbed andropause, subnormal androgen levels can lead to a decline in muscle mass, libido, sexual functioning and overall sense of well being later in life. Andropause is also commonly characterized by elevated estrogen to androgen to ratio, which is not surprising given that estrogen itself is suppressive of testosterone production in men. Elevated estrogen to androgen ratio is not only an important factor in andropause, but also in the development of a gynecomastia (female breast tissue development) condition. In many instances this indicates a need not only for some form of androgen replacement in later adulthood, but also some form of therapy to combat estrogenic activity.

A number of methods have been developed to mitigate the effects of estrogen in humans. The estrogen receptor antagonist tamoxifen for example, can drastically reduce the biological activity of serum estrogens by blocking their ability to interact with the appropriate receptor. Although still present in the body, estrogens are left will little ability to exert their effects. Clomiphene citrate is a chemically related selective estrogen-receptor modifier with similar activity in many human tissues, and has also been used effectively in men to block the effects of endogenous estrogen. Although these both are useful medications, it is more desirable in many circumstances to actually lower the level of estrogen in the blood instead of competitively inhibiting their activity however. This is most efficiently accomplished by direct inhibition of the aromatase enzyme, which is responsible for the formation of estrogens from androgen precursors.

Over the years numerous aromatase inhibitors have been developed. One such compound is 4-hydroxyandrostenedione. This compound is a 4-hydroxylated derivative of the natural hormone androstenedione, and has been shown to be a suicide substrate for the aromatase enzyme complex. This means that it irrevocably attaches the enzyme, preventing its ability to further interact with other aromatizable substrates. Studies have clearly supported the effectiveness of this compound, allowing it to be successfully used in many countries today. 4-hydroxyandrostenedione is in many regards close to an ideal method of dealing with estrogen in men suffering from andropause. It however could only be improved if a more active base steroid was used to create an aromatase inhibitor instead of the almost totally inactive androgen metabolite androstenedione.

### BRIEF SUMMARY OF THE INVENTION

The use of 4-hydroxyandrostenedione is a novel method of decreasing estrogen levels in humans. Although it can effectively be used to lower estrogen levels during andropause, it is not an ideal compound because 4-hydroxyandrostenedione is a modified form of an almost totally inactive androgen. The problem of the present invention is therefore to provide a modified form of an intrinsically active androgen, which will as effectively lower estrogen levels in humans. According to the invention this problem is solved by the use of 4-hydroxytestosterone. This compound is ideal because it is an active androgen, yet demonstrates an equally high level of efficacy in humans as an inhibitor of aromatase.

### BRIEF DESCRIPTION OF THE SEVERAL VIEWS OF THE DRAWING

Not Applicable

### DETAILED DESCRIPTION OF THE INVENTION

4-hydroxytestosterone (4,17b-dihydroxy-4-androstene-3-one) is a known androgen, shown in standard animal assays to exert similar but slightly diminished anabolic and androgen potency as its parent testosterone (G. Sala and G. Baldratti. Proc Soc. Exptl. Bio. Med. 95, 22 1957). Its use as an aromatase inhibitor however is a novel concept, and has beforehand never been proven or even suggested in the medical literature.

Tests carried out by Gual C, Morato T, Hayano M, Gut M and Dorfman R. (Endocrinology 71 (1962) 920-25) have suggested to this inventor that 4-hydroxytestosterone may indeed work just as well as 4-hydroxyandrostenedione at inhibiting aromatase in humans. This study did not look at either 4-hydroxylated steroid, but did discuss the ability of, among many other substrates, androstenedione and testosterone to aromatize to their corresponding estrogens. The interesting thing in this study is that both hormones aromatized with equal efficacy. It became apparent to this inventor that the change in the D ring of androstenedione from 17-keto to 17-beta hydroxyl did not effect its ability to bind aromatase. Furthermore, since androstenedione and testosterone are so closely related in structure and the modification at C-17 of androstenedione to form testosterone did not effect binding, it became possible to this inventor that a similar modification to 4-hydroxyandrostenedione to form 4-hydroxytestosterone would not effect aromatase interaction either. Furthermore it became the belief of this inventor that the same suicide-inhibitive effect can be achieved with 4-hydroxytestosterone.

It was the intention of this inventor to show that the advantages of 4-hydroxyandrostenedione as an aromatase inhibiting agent can also be achieved with 4-hydroxytestosterone. In an effort to prove this theory a clinical study was therefore undertaken by the inventor. Specifically it was the intention of the inventor to investigate whether 4-hydroxytestosterone would act as an effective in-vivo peroral aromatase inhibitor in man.

Oral 4-hydroxytestosterone can be given in daily doses of 25 mg. to 1000 mg.; preferably 100 to 500 mg. These daily doses can be divided into several subdoses with 3-5 being most preferable. In addition to peroral administration, 4-hydroxytestosterone can also be effectively administered by several other routes including injection, transdermal, intranasal and sublingual. A particular advantageous method of sublingual administration involves complexing 4-hydroxytestosterone with beta-hydroxypropyl-beta-cyclodextrin, which is then pressed into tablets. 4-hydroxytestosterone can also be modified at either C-4 or C-17 with a lipophilic ether such as tetrahydrophyranyl, cyclopentenyl or tetrahydrofuran to increase lymphatic system absorption and oral bioavailability. 4-hydroxytestosterone can also be modified at either C-4 or C-17 with a lipophilic ester derived from carboxylic acids with fewer than 9 carbon atoms to increase lymphatic absorption and oral bioavailability or to slow its release from an injection depot.

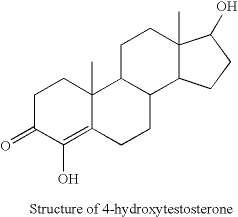

I claim:

1. A method of lowering the estrogen level in humans, said method comprising administering an effective amount of 4-hydroxytestosterone.

2. The method of claim 1 wherein said administration is peroral.

3. The method of claim 1 wherein said administration is selected from the group consisting of transdermal, intranasal, sublingual, ether-modified oral delivery, ester-modified oral delivery, or ester-modified injectable delivery.

4. The method of claim 1, wherein said amount is a daily dosage of 25 to 1000 mg.
